(19) United States
(12) Patent Application Publication
(10) Pub. No.: US 2019/0016520 A1
(43) Pub. Date: Jan. 17, 2019

(54) DISPOSABLE CONTAINER FOR SMALL PARTS IN THE PHARMACEUTICAL INDUSTRY

(71) Applicant: Eric NETZHAMMER, Arlesheim (CH)

(72) Inventor: Eric NETZHAMMER, Arlesheim (CH)

(21) Appl. No.: 15/777,631

(22) PCT Filed: Oct. 12, 2016
(86) PCT No.: PCT/EP2016/074394

§ 371 Date: May 18, 2018

(30) Foreign Application Priority Data
Oct. 20, 2015 (CH) 01528/15

(51) Int. Cl. B65D 81/20; B65D 88/22; B65D 90/00; B65D 90/12; B65D 90/48; A61L 2/08

(52) U.S. Cl. CPC B65D 81/2061; B65D 88/22; B65D 90/0033; A61L 2202/23; B65D 90/48; A61L 2/081; A61L 2202/181; B65D 90/12

(57) ABSTRACT

A container for filling, processing, transporting, storing, transferring, etc., of small parts for the pharmaceutical industry consists of a preformed, flexible container which has a predefined spatial shape and the form of which is changed by larger pressures due to its flexibility such that it may support enough pressure (e.g. 50-500 mbar) over a larger period (e.g. 1 year) in order to ensure the integrity of the content.

The invention concerns disposable containers for processing (sterilization, washing, drying, etc.), transporting, storing, filling containers with and transferal to a filling machine of small parts in the pharmaceutical industry and a method for handling flexible containers.

For the purpose of this description, some terms will be defined as follows:

A “filling machine” is a unit which fills active pharmaceutical ingredients or other substances into small units, e.g. vials, ampules or pre-filled syringes, etc.

Herein, the term “small parts” describes primary packaging components for substances which are used by a filling machine, e.g. as vial sealing plugs, etc.

In the pharmaceutical industry, small parts in the most diverse forms are required to close glass containers, to use as syringe components or for other applications. These small parts have to be cleaned, sterilized and dried, for example, before they are allowed to enter into use. This process (cleaning, sterilization, drying, etc.) is described as “handling” here. The parts named are “handled” when they are subjected to the handling process.

In this patent specification, the term “integrity” will be used similarly to in the pharmaceutical industry. “Integrity” is understood as when a container of any build is first sterilized internally and then exposed to excessive pressure without interruption. As long as the container is subject to excessive pressure, the container's contents count as sterile. This integrity is time-limited where necessary.

The term “Laminar Flow” describes a facility in which sterile air is led in a defined direction with a certain rate of flow.

The term “preformed flexible container” describes a container which, on the one hand, has a predefined form, as opposed to a foil bag or sack, and, on the other hand, whose form can be changed through high pressures, as opposed to a pressure container. An example of this kind of small container is a PET drinking bottle.

The small parts are currently either filled into pressure resistant containers, handled, stored, transported and transferred. In this case, there is a handling device in the pharmaceutical industry usually right next to a filling machine which further handles the small parts. Another possibility consists of acquiring and applying the small parts as “ready to use” parts on the market. These packaging units are usually small so that they can still be lifted by hand and connected to the filling machine. These packaging units are normally simple foil bags which can take no or little pressure. If a bag like this is damaged, it can hardly be seen. If such a packaging unit is exposed to a vacuum, there is a risk that the contents will be contaminated by the infiltration of a small amount of air without this being noticed. Large amounts of air would be noticed. In the pharmaceutical industry, it is customary to ensure the integrity of a container using excessive pressure. This is rarely the case for the currently available foil bags. The foil bags count as disposable containers. The port system which is often used to connect such bags to a filling machine is very expensive relative to the contents. As an alternative to the expensive disposable port systems, the bags can also be opened using a Laminar Flow. The effort it takes to maintain sterile conditions using this system is relatively large.

The invention is based on the task of making a container available which can take large amounts of small parts, can be used as a disposable container and can have a small amount of excess pressure applied to it in order to ensure the integrity, as well as proposing a method for handling flexible containers.

According to the invention, this is achieved using a container with features indicated in claim 1 as well as a method with the features indicated in claim 17. The following is a description of the exemplary embodiment of the invention with the help of the accompanying drawings. In the drawings:

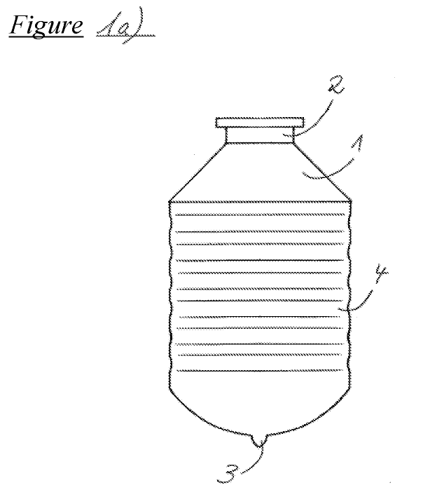

FIG. 1 shows an exemplary, preformed, flexible container,

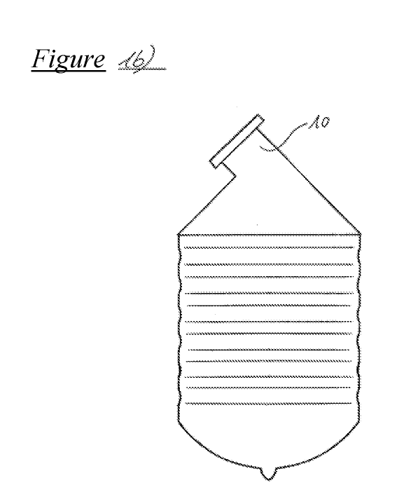

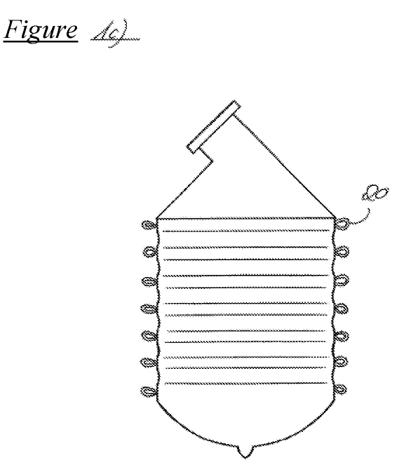

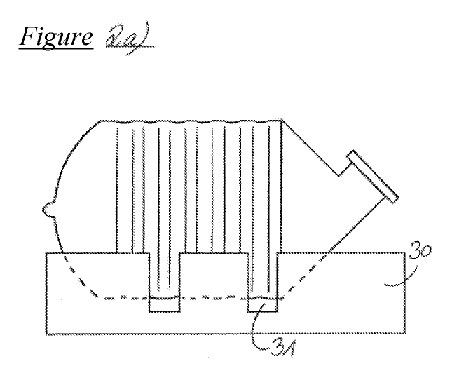

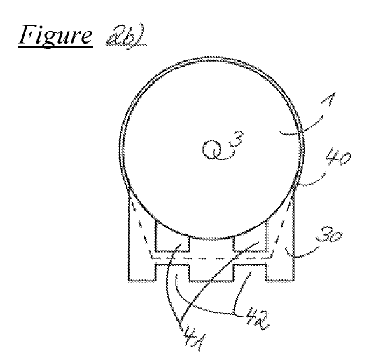

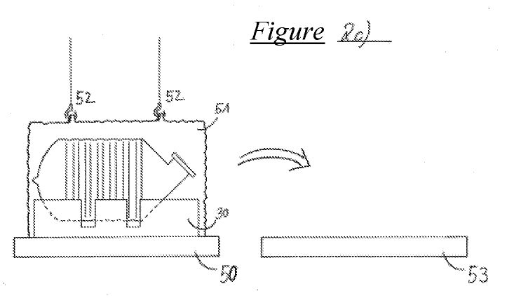

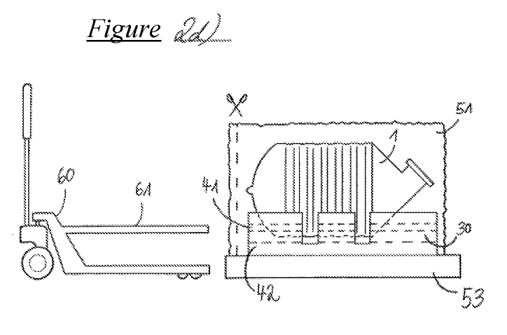

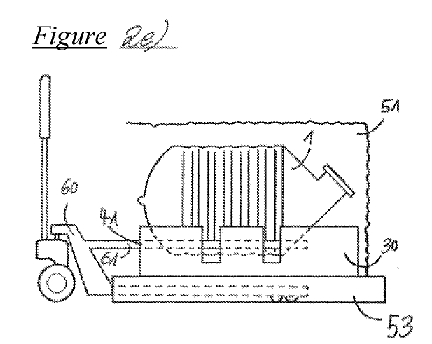

FIG. 2 shows the packaging, the transportation and the handling of a preformed flexible container within material locks and a clean room,

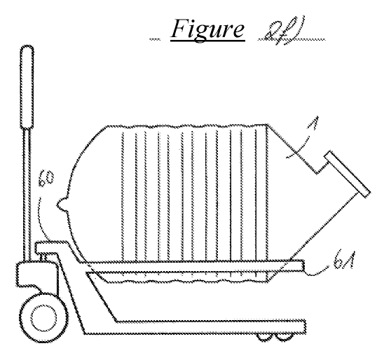

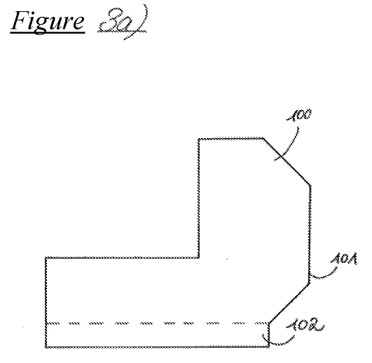

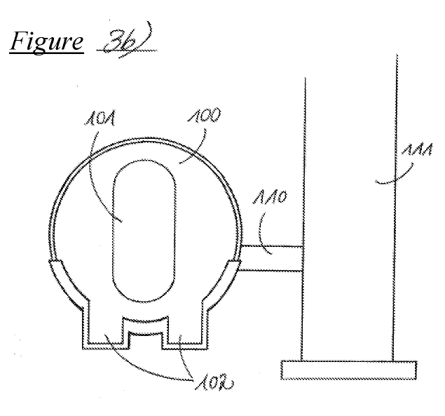

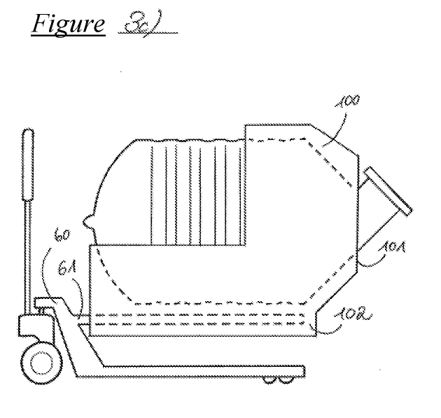

FIG. 3 shows an example for a lifting device on a filling machine in a clean room.

FIG. 1a shows a preformed, flexible container 1 with an opening with an interlocking/transfer system 2, a small fillet indentation 3 which is used to center any inserted ascension pipe during handling. The preformed, flexible container can be somewhat stabilized using small grooves on its circumference 4. Depending on its application, the container opening can be supplied with a flap so that the small parts do not fall out of the container during open transfer systems (the preformed, flexible container's opening usually points downwards). The flap is not included in the drawings.

Furthermore, it is possible to equip such a container with a piezochrome cell which changes color in relation to pressure. It can be determined by the color whether the container is still subject to pressure and the contents still have integrity.

FIG. 1b shows the container from FIG. 1a, but with a diagonally arranged opening.

FIG. 1c shows a container which is equipped with lugs or grommets 20 in order to be able to lift the container with a lifting device.

FIG. 2a shows a preformed, flexible container which is in a transport holder 30. The transport holder 30 consists of deep-drawn plastic, for example, and is manufactured so that the transport holder can carry a preformed, flexible container along with its contents. The transport holder has two or more lateral notches 31 so that the container can be lifted out of the transport holder with a lifting device. Such a lifting device can be placed on a filling machine, for example.

FIG. 2b shows a preformed, flexible container 1 along with a transport holder 30 from behind. The transport holder comes with two pairs of inlet openings 41 and 42, which can be driven into by a forklift truck. If the lower opening 42 is driven into, the transport holder with the container will be lifted. If the upper opening 41 is driven into, the container will be lifted out of the transport holder and will be in the forklift truck's fork. The container will be held in the transport holder by one or more tension belts 40 during transportation.

FIG. 2c. The preformed, flexible container is generally filled at one location but emptied at another location. The locations can be anywhere in the world. In order to fulfill the pharmaceutical industry's requirements, the preformed, flexible container with its transport holder is packed in a second lot of packaging, this is also called secondary packaging. High sanitary conditions prevail inside this secondary packaging. The container with its transport holder will be packed in the secondary packaging in a clean room immediately after the preformed, flexible container has been handled or filled. The secondary packaging can be a heat sealed bag made from an aluminum compound, for example. In order to balance out differences in pressure, it can be necessary to integrate a small disposable sterile filter into this secondary packaging (not shown in the Fig.). This secondary packaging is mostly also packaged in further packaging, e.g. cardboard box, plastic box or wooden box. The third lot of packaging (outer packaging) is not represented here. The packaging is generally transported on a commercially available wooden pallet. At the site of the goods recipient, typically in the logistics department, the secondary packaging with its entire content is lifted out of the outer packaging and thus from the wooden pallet and is re-palletized onto a clean pallet (e.g. aluminum or plastics). For lifting, lugs 52 may be mounted to the secondary packaging, for example. Alternatively, disposable belts may be placed around the secondary packaging. Usually, the secondary packaging will be cleaned on the outside. Only these clean pallets may enter the production building.

FIG. 2d/2e. In order to bring the secondary packaging with its content on a clean pallet into a clean room, the goods must pass a material lock. In the material lock, the secondary packaging 52 may be cut open with a pair of scissors or a knife. The fork 61 of a forklift truck 60 will be driven either into the openings 41 or the openings 42 of a clean pallet 53, and the container (and also the transport holder, if applicable) will be lifted. Then, the container (and the transport holder, if applicable) may be taken off the clean pallet 53 and the secondary packaging 51 may be removed. Depending on the consumer, the goods must pass one or two material transfer devices.

FIG. 2f. If two material locks are required, in the first transfer device, for example, as described in FIG. 2d/2e, the preformed, flexible container with its transport holder is taken off the secondary packaging, and in the second transfer device, the tension belts 40 are removed and the container is taken off the transport holder. The exact procedure is the responsibility of the consumer.

FIG. 3a/3b show a holding device 100 for a preformed, flexible container. The holding device is designed such that the preformed, flexible container fits into it. The holding device stabilizes the form of the preformed, flexible container. The holding device is provided with an opening 101, through which the opening of the preformed, flexible container may be pushed through by means of the transfer system. The holding device is designed such that the preformed, flexible container may not fall out downwards when the holding device is turned with its opening 101 facing downwards in order to couple to the filling machine.

As shown in FIG. 3c, the openings 102 enable to insert the hand operated forklift truck into the holding device, to lower the fork and thus to put down the preformed, flexible container in the holding device.

The holding device is, for example, attached to a lift 111 by means of a holder 110 which is used to lift and tilt the holding device 100 (and thus the preformed, flexible container) and couple it to the filling machine.

In order to be able to place the transfer system of the preformed, flexible container close enough to the transfer system of the filling machine, either the lift must perform the positioning as a function of the position of the transfer system of the preformed, flexible container, or the transfer system of the preformed, flexible container must be precisely positioned and fixed in the holding device 100, or the opening of the preformed, flexible container is movable enough to be able to compensate for tolerances occurring. A fixing device is not shown in FIG. 1. Those skilled in the art may easily provide the container with a groove, for example, and position the opening or the transfer system at the container, respectively, precisely in the holding device 100. If the opening or the transfer system of the container, respectively, is positioned precisely enough, the lift may be designed such that the preformed, flexible container may be coupled to the transfer system of the insulator with sufficient reproducibility.

In the context of the example embodiments described above, the transport and the unpacking of the flexible container are shown. Packing the container is performed almost in the same way, but vice versa. For filling such a container at a processing plant, a lift with a holding device may be used, as described in FIG. 3. This holding device needs an additional device such that the container does not fall out of the holding device, as the opening of the flexible container faces upwards when filling the flexible container. During draining, the opening faces downwards.

1. A container for filling, handling, transporting, storing, transferring, etc., of small parts for the pharmaceutical industry, characterized by a preformed, flexible container which has a predefined spatial form and the form of which is changed by larger pressures due to its flexibility such that it may support enough pressure (e.g. 50-500 mbar) over a larger period (e.g. 1 year) in order to ensure the integrity of the content.

2. The container according to claim 1, characterized in that the volume of such a container ranges from a few liters (e.g. 1 liter) up to sizes which can take a complete batch (e.g. 500 liters).

3. The container according to claim 1, characterized in that the volume of such a container is sufficient to be able to receive several batches of small parts.

4. The container according to claim 1, characterized in that the container is provided with a transfer system in order to be coupled to a filling machine.

5. The container according to claim 1, characterized in that the container is sterilized with gamma rays and then filled by means of a transfer system and exposed to an overpressure.

6. The container according to claim 1, characterized in that the container is cleaned, sterilized, dried, filled, etc., as a whole in a processing plant.

7. The container according to claim 1, characterized in that the container receives an ascension pipe for processing media in the event of processing within a processing plant or is permanently installed within it, through which the processing media are guided.

8. The container according to claim 1, characterized in that the container is packed in a closed bag (e.g. an aluminum compound bag) as a secondary packaging.

9. The container according to claim 1, characterized in that the container is put onto a transport holder in order to enable re-palletizing in a material lock.

10. The container according to claim 1, characterized in that the container is equipped with grommets, loops or similar in order to be able to lift the container at a filling machine.

11. The container according to claim 1, characterized in that the opening of the container is designed such that the opening is fixable with suitable holders defined.

12. The container according to claim 1, characterized in that the port connection at the container has enough mobility such that a precise positioning of the transfer connection to a filling machine is not required.

13. The container according to claim 1, characterized in that holding devices are used which may be, appropriately for the container, coupled to commercially available lift systems.

14. The container according to claim 1, characterized in that, additionally to the transfer system, another flap (usually a screen flap) is inserted in order to prevent the contents from falling out of the container when the transfer system is opened and the container is docked with another filling machine.

15. The container according to claim 1, characterized in that a film tube is inserted into the container in the area of the opening which is extendable and rotatable, wherein a rotation is for locking and the extended tube is used as a transport channel for the content.

16. The container according to claim 1, characterized in that the container is equipped with a piezochrome cell in order to be able to check the integrity based on the color.

17. A method for handling a flexible container for filling, processing, transporting, storing, transferring, etc., of small parts of the pharmaceutical industry, characterized in that the container is placed onto a holding device which enables lifting the container alone as well as together with the holding device using a lifting device.

18. The method according to claim 17, characterized in that the lifting device is a forklift truck.

19. The method according to claim 17, characterized in that the transport holder enables attaching the lifting device from different directions relative to the container.

20. The method according to claim 17, characterized in that the holding device stabilizes the container by means of its form adapted to the flexible container.

21. The method according to claim 20, characterized in that the holding device is lifted using a holder attached to a lift and that the transfer system of the flexible container is positioned at the transfer system of a filling device.
